(19) United States
(12) Patent Application Publication
(10) Pub. No.: US 2008/0152633 A1
(43) Pub. Date: Jun. 26, 2008

(54) Flavivirus Replicon Constructs for Tumor Therapy

(75) Inventors: Andreas Suhrbier, Bunya Queensland (AU); Alexander A. Khromykh, THE GAP Queensland (AU)

Correspondence Address: HOFFMANN & BARON, LLP, 6900 JERICHO TURNPIKE, SYOSSET, NY, 11791, US

(73) Assignees: THE UNIVERSITY OF QUEENSLAND, Brisbane Queensland (AU); The Council of the Queensland Institute of Medical, Herston, Queensland (AU)

(21) Appl. No.: 11/816,350

(22) PCT Filed: Feb. 16, 2006
(86) PCT No.: PCT/AU06/00198

§ 371 Date: Aug. 15, 2007

(30) Foreign Application Priority Data
Feb. 16, 2005 (AU) 2005900714
Feb. 16, 2006 (AU) PCT/AU2006/000198

(51) Int. Cl. A61K 48/00; C07H 21/00; C12N 15/09; A61P 35/04; C12N 5/06; A61K 31/7088

(52) U.S. Cl. 424/93.21; 536/23.72; 435/320.1; 435/325; 514/44

(57) ABSTRACT

A flaviviral replicon-based construct is provided for delivery and expression of granulocyte-macrophage colony stimulating factor to facilitate tumour therapy. In particular, the replicon construct encodes a Kunjin virus replicon having one or more mutations in an NS2A non-structural protein that induce enhanced levels of cellular IFN that synergize with recombinant granulocyte-macrophage colony stimulating factor delivered according to the invention. The construct may be administered intra-tumourally or peri-tumourally to an animal as DNA, RNA or packaged into a VLP, for the therapeutic and/or prophylactic treatment of tumours and cancers such as melanoma, lung carcinoma, cervical carcinoma, lung epithelial carcinoma, prostate cancer, breast cancer, renal carcinoma, colon cancer, epithelial cancers and mesothelioma.

### FIELD OF THE INVENTION

THIS INVENTION relates to a flaviviral replicon-based expression construct for delivery and expression of granulocyte-macrophage colony stimulating factor (GMCSF). More particularly, this invention relates to a Kunjin virus replicon-based expression construct for delivery and expression of GMCSF for tumour therapy.

### BACKGROUND OF THE INVENTION

GMCSF is a potentially useful cytokine for cancer treatment. For example, B16 melanoma cells made to express recombinant GMCSF following transfection were able to be used as live, whole cell vaccines when irradiated and injected into a naïve mice. Such vaccinated mice were protected against subsequent challenge with B16. These initial prophylactic murine experiments have led to human therapeutic cancer trials, which have used the same principle.

Vaccination with irradiated melanoma cells engineered to secrete GMCSF enhances the host's immune responses through improved tumour antigen presentation by recruited dendritic cells and macrophages. This results in the induction of cancer specific CD8 T cells, which attack the cancer (Dranoff, 2003, Oncogene 22 3188-92.). Such whole cell vaccination strategies are complicated by the need to generate and inoculate live transfected tumour lines as vaccines into the patient (Ellem et al., 1997, Cancer Immunol Immunother. 44 10-20). A substantial number of vaccine modalities, which exploit the properties of GMCSF have been investigated (Chang et al., 2004, Hematology 9 207-15).

Of these approaches, the area of potentially greatest utility has been the direct intra-tumoural and/or peri-tumoural injection of viral vectors capable of infecting cancer cells and/or surrounding cells and causing those cells to produce recombinant GMCSF. Such approaches do not require the ex vivo generation of cells and have shown some promise in tumour therapy for a number of different cancers (Triozzi et al., Int J. Cancer. 2004 28; Yang et al., 2003, Cancer Res. 63 6956-61; Parkinson et al., 2003, Prostate 56 65-73; Pan et al., 2004, Cancer Immunol Immunother. 53 17-25).

While promising, current systems do not appear capable of reliably curing tumours. Accordingly, many in the field are seeking to improve tumour therapies by exploiting synergies with other anti-cancer modalities. However, these approaches have typically been undertaken on a “trial and error” basis, as a predictive science has yet to emerge.

### OBJECT OF THE INVENTION

It is therefore an object of the invention to provide an improved tumour therapy system that utilizes delivery of GMCSF.

### SUMMARY OF THE INVENTION

The present inventors have recently discovered that delivery of GMCSF using a flavivirus replicon expression construct, such as but not limited to a Kunjin virus replicon expression construct, is particularly efficacious in GMCSF-mediated tumour therapy. More particularly, Kunjin virus replicon-containing constructs having mutations in replicon-encoded non-structural proteins, such as but not limited to NS2A, are particularly efficacious, perhaps through an ability to induce enhanced levels of IFNα/β secretion and/or other inflammatory cytokines that synergize with recombinant GMCSF to enhance tumour therapy.

Thus, the invention is broadly directed to delivery of GMCSF, using a flavivirus replicon-containing construct, such as but not limited to a Kunjin virus replicon construct, for the purpose of prophylactic or therapeutic treatment of tumours or cancers.

In a first aspect, the invention provides a flavivirus replicon construct comprising a nucleotide sequence encoding:

(i) a flavivirus replicon that is incapable of producing infectious virus; and

(ii) granulocyte macrophage colony stimulating factor (GMCSF).

The flavivirus replicon construct may be in the form of RNA or DNA.

In a preferred embodiment, the nucleotide sequence encodes a flavivirus replicon having one or more amino acid mutations, deletions or substitutions in a non-structural protein of said replicon.

Preferably, said non-structural protein is selected from the group consisting of: NS2A, NS2B, NS3, NS4A and NS4B.

Preferably, said one or more amino acid mutations, deletions or substitutions in a flaviviral non-structural protein is selected from the group consisting of:

(I) a nonstructural protein NS2A having a mutation of Alanine 30 to Proline; and

(II) a nonstructural protein NS2A having a mutation of Asparagine 101 to Aspartate or Glutamate.

The invention also contemplates one or more other amino acid mutations, deletions or substitutions in one or more respective non-structural proteins of said replicon, which in an animal cell, enhance induction of IFNα/β or other proinflammatory cytokines or chemokines compared to a wild-type flavivirus replicon-encoded non-structural protein.

In a preferred embodiment, the flavivirus replicon construct encodes a Kunjin virus replicon.

In a second aspect, the invention provides an expression construct comprising the flavivirus replicon construct of the first aspect operably linked to one or more regulatory sequences.

Preferably, in cases where the expression construct is DNA, the one or more regulatory sequences include a promoter.

In embodiments where the expression construct is a DNA construct for the transcription of flavivirus replicon-encoding RNA in vitro, the promoter may be an SP6 or T7 promoter, although without limitation thereto.

In embodiments where the expression construct is a DNA construct for expression in an animal cell, the promoter is suitably operable in said animal cell to facilitate expression of a flavivirus replicon-encoding RNA by said animal cell.

In a third aspect, the invention provides an expression system comprising:

(i) a DNA or RNA expression construct according to the second aspect; and

(ii) a packaging construct that is capable of expressing one or more proteins that facilitate packaging of said expression vector or construct into flavivirus virus like particles (VLPs) by said packaging cell.

Preferably, the expression construct in (i) is RNA.

Although VLP production by a packaging cell preferably utilizes flavivirus replicon-encoding RNA transcribed in vitro, alternative embodiments contemplate a DNA expression construct for transfection into a packaging cell for production of VLPs. According to this alternative embodiment the promoter is suitably operable in the packaging cell to facilitate expression of a flavivirus replicon-encoding RNA by the packaging cell.

In a preferred form of this aspect, the packaging system in (ii) comprises a regulatable promoter, such as a tetracycline-regulatable promoter In a particularly preferred from, the packaging construct comprises a regulatable promoter operably linked to a nucleotide sequence encoding a flavivirus structural protein translation product, which comprises C protein, prM protein and E protein.

In a fourth aspect, the invention provides a flavivirus virus like particle (VLP) comprising the replicon construct of the first aspect in RNA form.

In a fifth aspect, the invention provides a packaging cell comprising the expression system of the third aspect.

In a sixth aspect, the invention provides a pharmaceutical composition comprising an RNA replicon construct of the first aspect, a DNA expression construct of the second aspect, or a flavivirus virus like particle (VLP) of the fourth aspect together with a pharmaceutically-acceptable carrier, diluent or excipient.

In a seventh aspect, the invention provides a method of prophylactic or therapeutic treatment of a tumour or cancer in an animal, said method including the step of administering an RNA replicon construct of the first aspect, a DNA expression construct of the second aspect, or a flavivirus virus like particle (VLP) of the fourth aspect to an animal to thereby reduce, arrest, eliminate or otherwise treat the tumour or cancer in said animal.

Preferably, said method includes the step of administering the pharmaceutical composition intra-tumourally or peri-tumourally.

It will also be appreciated that the method of the invention may be used as a combination therapy with at least one other tumour or cancer therapy, such as but not limited to a tumour or cancer immunotherapy or cancer vaccine.

In an eighth aspect, the invention provides an isolated cell that is obtained from an animal treated according to the seventh aspect.

Preferably, the isolated cell is an immune cell such as an antigen presenting cell, lymphoid or myeloid or other cell that is a component of an animal immune system.

In one particular embodiment, the isolated cell is an antigen-presenting cell, such as a dendritic cell.

In another particular embodiment, the isolated cell is a lymphocyte, such as a tumour-specific T lymphocyte.

It will be appreciated that such cells may have particular efficacy in adoptive immunotherapy of a tumour.

According to the aforementioned aspects, animals include humans, domestic livestock, companion animals, poultry and any other animals of commercial importance, although without limitation thereto.

Preferably, the animal is a mammal.

More preferably, the animal is a human.

Non-limiting examples of tumours or cancers that may be treated according to the invention include melanoma, lung carcinoma, cervical carcinoma, lung epithelial carcinoma, prostate cancer, breast cancer, renal carcinoma, colon cancer, epithelial cancers and mesothelioma, although without limitation thereto.

Throughout this specification, unless otherwise indicated, “comprise”, “comprises” and “comprising” are used inclusively rather than exclusively, so that a stated integer or group of integers may include one or more other non-stated integers or groups of integers.

### BRIEF DESCRIPTION OF THE FIGURES

FIG. 1. Kaplan Meier plot of survival. B16 tumours were established on groups of C57BL6 mice (n=6 per group except n8 for Control). The tumours were treated d0, d1, d2, d6, d7 and d 8 intratumourally/peritumourally (i.t./p.t). with nothing (Control), RPMI1640/10% FCS (Medium Control), KUN VLP Control or KUN VLP GMCSF.

FIG. 2. Growth curves for the same experiment shown in FIG. 1.

FIG. 3. Kaplan Meier plot of survival.

FIG. 4. Growth curves for the same experiment shown in FIG. 3. Lines terminate on the day the first animal in the group was culled as tumour size reached 10×10.

FIG. 5. Kaplan Meier plot of survival. Treatment ceased on d 9.

FIG. 6. Growth curves for the same experiment shown in FIG. 5. Lines terminate on the day the first animal in the group was culled as tumour size reached 10×10, except for the KUN VLP GMCSF+KUN VLP mpt group, where no animals in the group were culled on or before d 33. The number of animal without visible tumour is indicated for each group at the time when the first animal in the group was culled, except for the KUN VLP GMCSF+KUN VLP mpt group where no animals were culled and no tumours were visible on d 33.

FIG. 7. (A) Growth curves of mean tumour size for sc AE17 tumours treated with and without i.t./p.t. KUN VLP GMCSF. (B) Kaplan Meier plot of survival. Treatment ceased on d9.

FIG. 8. (A) Growth curves of mean tumour size for sc MC38 tumours treated with and without i.t./p.t. KUN VLP GMCSF. (B) Kaplan Meier plot of survival. Treatment ceased on d9.

FIG. 9. (A & B) Individual growth curves of tumour size for each sc TUBO tumour for treated (Group 1 mice M1-M4; A) and untreated (Group 2 mice M1-M5; B) mice. (C) Kaplan Meier plot of survival. TUBO tumours were established on groups of balb/c mice (n=4 for Test, n=5 for Control). The tumours were treated d0 to d9 i.t./p.t with nothing (Control) or Kun VLP GMCSF.

FIG. 10. (A) Growth curves of mean tumour size for sc 4T1 tumours treated with and without i.t./p.t. KUN VLP GMCSF. (B) Kaplan Meier plot of survival.

FIG. 11. GMCSF production by BHK cells transfected with KUN GMCSF RNA.

FIG. 12. Detection of IFN-β mRNA and of secreted IFN-α/, in A549 cells infected with the wild type and NS2A-mutated KUN viruses. (A) Northern blot analysis of A549 cells infected for 24 h with MOI=1 of KUN virus encoding the wild type NS2A (wtNS2A) or with MOI=3 of KUN virus encoding Ala30 to Pro-mutated NS2A (NS2A/A30P) genes. The probes were specific for KUN RNA, IFN-β mRNA, and β-actin mRNA. (B) Bioassay analysis of 24h culture fluid from the same infected A549 cells. New A549 cells were incubated with collected culture fluids for 24 h and then infected with 0.5 MOI of Semliki Forest virus (SFV). The IFN α/β production was estimated by the protection of cells from cytopathic effect of SFV infection and calculated relative to the protection afforded by the reference IFN-2a (Sigma) with known biological activity.

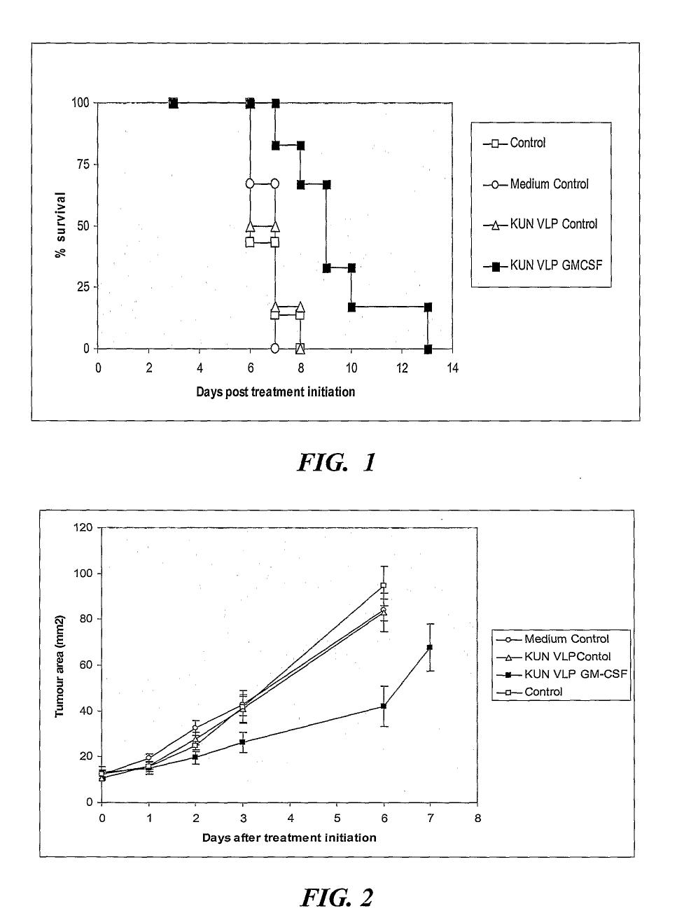

### DETAILED DESCRIPTION OF THE INVENTION

The present invention arises, at least in part, from the present inventors' recognition of the role of IFNα/β as a link between the innate and adaptive immune system and the ability of cellular and/or secreted IFNα/β to synergize with recombinant GM-CSF to cause both recruitment and activation of dendritic cells.

More particularly, Kunjin virus VLPs comprising a Kunjin virus replicon-containing construct that encodes GMCSF and having a mutation in NS2A, caused tumour growth to arrest in mice injected intratumourally for 8-10 days with the Kunjin VLPs. Control tumours grew rapidly within this time frame requiring that the animals be euthanased.

Although not wishing to be bound by any particular theory, the present inventors believe that Kunjin virus replicon-containing vector-induced IFNα/β may synergize with recombinant GMCSF to cause both recruitment and activation of dendritic cells, which facilitate the arrest in tumour growth.

Additional contributing factors may be the secretion of other cytokines or chemokines, and the well described persistent non cytopathic nature of Kunjin replicons, plus their ability to pass genetic material to both daughter cells following replication of a transfected cell. The latter features may promote sustained release of GMCSF.

More particularly, it appears that Kunjin replicons having mutations in non-structural proteins such as NS2A, induce enhanced levels of cellular IFNα/β that synergize with recombinant GMCSF delivered according to the invention.

### Flavivirus Replicon Constructs

One aspect of the invention provides a flavivirus replicon construct comprising a nucleotide sequence that encodes:

(i) a flavivirus replicon that is incapable of producing infectious virus; and

(ii) granulocyte macrophage colony stimulating factor (GMCSF).

In another aspect, the invention provides an expression construct comprising the aforementioned replicon construct operably linked to a promoter and one or more other regulatory sequences.

Thus the invention provides nucleic acid constructs that may be used to facilitate expression of a GMCSF protein, such as for the purposes of tumour therapy.

The term “nucleic acid” as used herein designates single- or double-stranded mRNA, RNA, cRNA and DNA inclusive of cDNA and genomic DNA.

By “protein” is meant an amino acid polymer. Amino acids may include natural (i.e genetically encoded), non-natural, D- and L-amino acids as are well known in the art.

A “peptide” is a protein having less than fifty (50) amino acids.

A “polypeptide” is a protein having fifty (50) or more amino acids.

The nucleotide sequence encoding GMCSF may encode any form of GMCSF that assists, augments, enhances or otherwise facilitates tumour therapy in an animal, particularly in a human.

It will therefore be appreciated that for tumour therapy in a human, said nucleotide sequence preferably encodes a human GM-CSF protein.

The invention also contemplates nucleotide sequences encoding biologically-active fragments of GMCSF protein, and/or variants of a GM-CSF protein.

Suitably, biologically-active fragments and/or variants of GM-CSF have at least 25%, preferably at least 50%, more preferably at least 75% or even more preferably at least 80%, 90%, 95% or 100% of the biological activity of full length or wild type GM-CSF.

Suitably, variants of GM-CSF have at least 75%, preferably at least 80%, more preferably at least 85% or even more preferably at least 90%, 95% or 98% sequence identity with wild type GM-CSF.

Sequence identity may be conveniently measured by programs such as BLASTP and CLUSTALW which are well known in the art.

As used herein, “flavivirus” and “flaviviral” refer to members of the family Flaviviridae within the genus Flavivirus, which contains 65 or more related viral species. Typically, flavivirus are small, enveloped RNA viruses (diameter about 45 nm) with peplomers comprising a single glycoprotein E. Other structural proteins are designated C (core) and M (membrane-like). The single stranded RNA is infectious and typically has a molecular weight of about 4×10⁶ with an m7G ‘cap’ at the 5′ end but no poly(A) tract at the 3′ end; it functions as the sole messenger. Flaviviruses infect a wide range of vertebrates, and many are transmitted by arthropods such as ticks and mosquitoes, although a separate group of flaviviruses is designated as having no-known-vector (NKV).

Particular, non-limiting examples of flavivirus are West Nile virus inclusive of NY99 strain, Kunjin virus, Yellow Fever virus, Japanese Encephalitis virus, Dengue virus, Montana Myotis leukoencephalitis virus, Usutu virus, St Louis Encephalitis virus and Alkhurma virus.

The West Nile virus subgroup somewhat controversially includes Kunjin virus as a sub-type. Nevertheless, according to the present specification Kunjin virus and West Nile virus are considered to be distinct flaviviruses.

Although the present invention has primarily been exemplified using Kunjin virus replicon-containing expression constructs, it is contemplated that the inventive principle described herein may be extendible to other flavivirus replicon-containing constructs.

It is also contemplated that a flavivirus replicon construct derived from one particular flavivirus may be packaged into VLPs of another particular flavivirus. In this regard, data are presented hereinafter that demonstrate Kunjin virus VLPs containing a West Nile virus replicon construct.

Kunjin virus replicons contemplated by the present invention include any self-replicating component(s) derivable from Kunjin virus RNA as described hereinafter and, for example, in International Publication WO 99/28487, International Publication WO 03/046189 and Varnavski et al., 2000, J. Virol. 74 4394-4403.

As generally used herein, flavivirus replicons are derived from flavivirus or are otherwise of flavivirus origin. Thus, in the context of this specification “a nucleotide sequence encoding a flavivirus replicon” is a DNA or RNA sequence that comprises sequence information from a flavivirus replicon or at least a portion thereof sufficient for replication while being incapable of producing infectious virus.

For example, as will be understood by persons skilled in the art, DNA-based constructs of the invention referred to herein comprise a DNA copy of replicon RNA, which is complementary to or otherwise derived from said replicon RNA.

Suitably, the flavivirus replicon is replication competent while being “incapable of producing infectious virus”. By this is meant that the flavivirus replicon is unable to express one or more structural proteins either in their entirety or in part, that are required for viral packaging. A detailed description of modifications to Kunjin flaviviral replicons to disable viral packaging is provided in International Publication WO 99/28487.

In a preferred embodiment, the flavivirus replicon further comprises:

(i) 5′ and 3′ untranslated (UTR) sequences and sequences encoding the first 20 amino acids of C protein (C20) and the last 22 amino acids of E protein (E22) respectively; and

(ii) nucleotide sequence encoding nonstructural proteins NS1, NS2A, NS2B, NS3, NS4A, NS4B and NS5.

In a more preferred embodiment, one or more of said nonstructural proteins encoded by the replicon comprises an amino acid sequence mutation, deletion or substitution which in an animal cell, enhances induction of IFNoC/P compared to a wild-type flavivirus replicon.

Preferably, said non-structural protein is selected from the group consisting of: NS2A, NS2B, NS3, NS4A and NS4B. In one particular embodiment, alanine 30 of the Kunjin NS2A protein is substituted by proline.

In another particular embodiment, asparagine 101 of the Kunjin NS2A protein is substituted by aspartate or glutamate.

It will also be appreciated that each of the above mutations or substitutions may be present individually or in combination in a flavivirus replicon of the invention.

The invention also contemplates one or more other amino acid mutations, deletions or substitutions in a non-structural protein of said replicon, which in an animal cell, enhances induction of IFNα/β compared to a wild-type flavivirus replicon.

According to the present invention, an “expression construct” comprises a flavivirus replicon construct of the first aspect operably linked to one or more regulatory sequences.

According to one embodiment of the present invention, an expression construct is an RNA construct that facilitates expression of a recombinant GMCSF protein, or a biologically active fragment thereof, in a mammalian cell.

According to another embodiment of the present invention, an expression construct is DNA construct that facilitates transcription of a flavivirus replicon RNA from the DNA construct in a mammalian cell, thereby facilitating expression of a recombinant GMCSF protein, or a biologically active fragment thereof, in the mammalian cell.

In yet another embodiment, an expression construct is a DNA construct that facilitates transcription of flavivirus replicon construct RNA from the DNA construct in vitro.

In still yet another embodiment, an expression construct is a DNA construct that facilitates transcription of a flavivirus replicon construct RNA from the DNA construct in a packaging cell, thereby facilitating production of VLPs by the packaging cell.

According to the present invention an expression construct further comprises one or more other regulatory nucleotide sequences. Such regulatory sequences include but are not limited to a promoter, internal ribosomal entry site (IRES), restriction enzyme site(s) for insertion of one or more heterelogous nucleic acid(s), foot and mouth disease virus 2A autoprotease sites, polyadenylation sequences and other sequences such as an antigenomic sequence of the hepatitis delta virus ribozyme (HDVr) that ensure termination of transcription and precise cleavage of 3′ termini, respectively.

A DNA expression construct of the invention suitably comprises a promoter operably linked to the flavivirus replicon construct.

By “operably linked” or “operably connected” is meant that said promoter is positioned to initiate, regulate or otherwise control in vitro or intracellular transcription of RNA encoding said flavivirus replicon and any other regulatory sequences present that facilitate RNA processing and protein expression.

Preferably, the promoter is located 5′ of the flavivirus replicon.

A preferred promoter for in vitro transcription of RNA from a DNA expression construct is an SP6 promoter.

A preferred promoter for intracellular transcription of RNA from a DNA expression construct in an animal cell (e.g. in a mammalian cell such as a packaging cell line or following therapeutic administration to an animal) is a cytomegalovirus (CMV) promoter. However, it will be appreciated that other well-known promoters active in mammalian cells are contemplated, including an SV40 promoter, a human elongation factor alpha promoter and an alpha crystallin promoter, although without limitation thereto.

### Viral Packaging and VLP Production

According to the third-mentioned aspect of the invention, there is provided a flaviviral expression system comprising:

(i) a DNA or RNA expression construct according to the second aspect that comprises a promoter operable in a packaging cell; and

(ii) a packaging construct that is capable of expressing one or more proteins that facilitate packaging of said expression vector or construct into flavivirus virus like particles (VLPs).

It will be appreciated that in certain broad embodiments, flaviviral packaging may be achieved by:

(a) transient transfection of host cells (such as hereinbefore described) with a flaviviral expression construct encoding GMCSF and a packaging construct that provides structural proteins required for viral packaging;

(b) transient transfection of host cells with a flaviviral expression construct encoding GMCSF, wherein the host cells have been stably transfected with a packaging construct that provides structural proteins required for viral packaging.

With regard to (a), the expression and packaging constructs may be co-transfected or may be separately transfected within a time frame that allows optimal VLP production.

With regard to the above, “transfected” is used for convenience as a general term encompassing transient or stable introduction of foreign genetic material into a host cell.

Transfection of packaging cells may be achieved by methods well known in the art such as calcium phosphate precipitation, electroporation, lipofectamine, lipofectin and other lipophilic agents, calcium phosphate precipitation, DEAE-Dextran, microparticle bombardment and microinjection.

Preferably, although not exclusively, the flavivirus expression construct in (i) is RNA transcribed in vitro from a DNA expression construct of the invention and transfected into a packaging cell.

Although VLP production by a packaging cell preferably utilizes flavivirus replicon-encoding RNA transcribed in vitro, alternative embodiments contemplate a DNA expression construct for transfection into a packaging cell for production of VLPs. According to this alternative embodiment the promoter is suitably operable in the packaging cell to facilitate expression of a flavivirus replicon-encoding RNA by the packaging cell.

In a particularly preferred form, the invention contemplates transient transfection of packaging cells with a flavivirus expression construct RNA encoding GMCSF, wherein the packaging cells have been stably transfected with a packaging construct that provides structural proteins required for viral packaging.

Preferably, the promoter of the packaging construct is a regulatable promoter, such as a tetracycline-regulatable promoter.

In a particularly preferred from, the packaging construct comprises a regulatable promoter operably linked to a nucleotide sequence encoding a flavivirus structural protein translation product, which comprises C protein, prM protein and E protein.

For the purposes of generating stably-transformed packaging cells, the packaging construct further comprises a selectable marker gene. Selectable marker genes are well known in the art and include neomycin transferase and puromycin N-acetyl transferase, without limitation thereto.

With regard to packaging constructs for regulatable expression of structural proteins, reference is made to International Publication WO2004/108936, which provides a detailed disclosure in relation to the production and use of regulatable expression of Kunjin virus structural proteins by stably-transfected packaging cells, the entirety of which is incorporated herein by reference.

It will also be appreciated that alternatively, other vectors may be used for expression of flaviviral structural proteins in production of VLPs. For example, said packaging construct could be derived from alphavirus, such as Semliki Forest virus (SFV) or Sindbis virus (SIN) or from DNA viruses such as adenovirus, fowlpox virus or vaccinia virus.

Examples of SFV-derived packaging constructs are provided in International Publication WO 99/28487 and International Publication WO 03/046189.

Suitable packaging cells may be any eukaryotic cell line that is competent to effect transcription, translation and any post-transcriptional and/or post-translational processing or modification required for protein expression and VLP production. Examples of mammalian cells typically used for nucleic acid transfection and protein expression are COS, Vero, CV-1, BHK21, HEK293, Chinese Hamster Ovary (CHO) cells and NIH 3T3, Jurkat, WEHI 231, HeLa MRC-5, and B16 melanoma cells, although without limitation thereto.

Preferred the packaging cells are BHK21 cells.

### Pharmaceutical Compositions and Methods of Tumour or Cancer Therapy

A particular aspect of the invention relates to use of a flaviviral replicon construct that encodes GMCSF in the therapeutic and/or prophylactic treatment of tumours.

Pharmaceutical compositions for delivery of GMCSF-encoding replicon constructs according to the invention may comprise:

(i) RNA-containing VLPs;

(ii) “naked” RNA transcribed in vitro from a DNA expression construct of the invention; or

(iii) a plasmid DNA expression construct of the invention capable of directing transcription of RNA in vivo.

Preferably, but not exclusively, pharmaceutical compositions according to the invention of the invention comprise RNA-containing VLPs.

The pharmaceutical composition may further comprise a pharmaceutically-acceptable carrier, diluent or excipient.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991) which is incorporated herein by reference.

Any safe route of administration may be employed for providing a patient with the composition of the invention. For example, oral, rectal, parenteral, sublingual, buccal, intravenous, intra-articular, intramuscular, intra-dermal, subcutaneous, inhalational, intraocular, intraperitoneal, intracerebroventricular, transdermal and the like may be employed. Intra-muscular and subcutaneous injection is appropriate, for example, for administration of immunotherapeutic compositions, proteinaceous vaccines and nucleic acid vaccines.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Pharmaceutical compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

In embodiments relating to delivery of “naked” DNA or RNA expression constructs of the invention in vivo, the pharmaceutically acceptable carrier diluent or excipient may be an agent that specifically facilitates RNA or DNA delivery.

By way of example, lipophilic agents such as, but not limited to, cationic liposomes have been successfully used to deliver nucleic acids in vivo. A more recent cationic liposome has been developed based on a synthetic cationic cardiolipin analogue (CCLA) for this purpose.

In a preferred form, the pharmaceutical composition of the invention is administered intra-tumourally and/or peri-tumourally.

Tumours and cancers that may be treated according to the invention include melanoma, lung carcinoma, cervical carcinoma, lung epithelial carcinoma, prostate cancer, breast cancer, renal carcinoma, colon cancer, epithelial cancers and mesothelioma, although without limitation thereto.

The therapeutic methods and compositions of the invention may be administered alone or as an adjunct therapy in combination with other treatments such as chemotherapy, radiation therapy, immune-based therapies such as cancer vaccines or cytokine therapy.

In this regard, an example is provided hereinafter where a Kunjin VLP encoding a murine polytope (KUN VLP mpt) that includes the ovalbumin epitope, SIINFEKL (SEQ ID NO:1; Anraku et al., 2002, supra) synergized with a Kunjin virus VLP encoding GMCSF.

Reference is also made to Wei et al., 2005, Cell. Mol. Immunol. 2 351, which provides a current review of cancer immunogene therapy that may provide guidance to persons skilled in the art.

In one particular embodiment, the invention contemplates transfecting an autologous tumour cell in vitro so that the tumour cell expresses an immunologically active cytokine (typically but not exclusively GMCSF) and using the transfected cell as an anti-tumour vaccine (as for example described in Ellem et al., 1997, supra) in conjunction with Kunjin replicon GMCSF therapy according to the invention.

In another particular embodiment, the invention contemplates isolation of dendritic cells or their bone marrow precursors, transfection of said dendritic cells with a tumour antigen and administration of the transfected dendritic cells to said animal (as for example described in Metharom et al., 2005, Cell. Mol. Immunol. 2 281) in conjunction with Kunjin replicon GMCSF therapy according to the invention.

In yet another embodiment, the invention contemplates combining Kunjin replicon GMCSF therapy with other immune based therapies such as adoptive transfer of autologous in vitro generated tumour- and/or cancer-specific T cells or with anti-cancer antibodies (e.g. herceptin).

In light of the foregoing, it will be appreciated that according to the aforementioned aspects, animals include humans, domestic livestock, companion animals, poultry and any other animals of commercial importance, although without limitation thereto.

Preferably, the animal is a mammal.

More preferably, the animal is a human.

It will also be appreciated that the invention provides an isolated cell that is obtained from an aforementioned animal treated according to the invention.

Although not wishing to be bound by any particular theory, it is contemplated that immune cells isolated from an animal treated according to the invention may have improved immunotherapeutic properties compared to cells obtained from untreated animals.

In one particular embodiment, the isolated cell is an antigen-presenting cell, such as a dendritic cell or a dendritic cell precursor, such as a CD14⁺ monocyte, as for example described in Curti et al., 2004, Leuk. Lymphoma 45 1419-1428 and/or Babatz et al., 2003, J Hematother Stem Cell Res. 12 515-23.

Also contemplated according to this embodiment is isolation of dendritic cells, or their bone marrow precursors, from an animal treated according to the invention, transfection of said dendritic cells with a tumour antigen and administration of the transfected dendritic cells to said animal to thereby reduce, arrest, eliminate or otherwise treat the tumour in said animal.

In another particular embodiment, the isolated cell is a tumour-specific T lymphocyte inclusive of CD8⁺ or CD4⁺CTL and/or helper T cells, suitable for adoptive immunotherapy such as reviewed in Yamaguchi et al., 2003, Hum Cell 16 183-9, for example.

So that the invention may be readily understood and put into practical effect, the skilled person is directed the following non-limiting examples.

### EXAMPLES

### Construction of KUN Replicons Expressing Murine GMCSF and Production of Replicon VLPs

Kunjin replicon Sp6KUNrep4 was made by replacing the CMV promoter of Kunjin replicon pKUNrep4 (Varnavski et al., 2000, J Virol 74, 4394-4403) with the SP6 promoter, so that RNA could be transcribed in vitro by SP6 RNA polymerase. Sp6KUNrep4 encodes a puromycin-selection marker, a foot and mouth disease virus (FMDV) 2A autoprotease to cleave off the inserted heterologous protein at the N-terminus, and contains an Encephalomyocarditis virus (EMCV) internal ribosomal entry site (IRES), which initiates the translation of the KUN nonstructural genes required for RNA replication. The IRES also allows for the stop codon of the heterologous gene to be maintained, ensuring the production of heterologous protein with an authentic C-terminus.

To further enhance persistent RNA replication in cells, a cell line-adaptive mutation was subsequently introduced into Sp6KUNrep4. This specific mutation in NS2A at amino acid position 30 (Ala30 to Pro) resulted in 15- to 50-fold more efficient establishment of persistent replication in hamster (BHK21) and human (HEK293 and HEp-2) cell lines (Liu et al., 2004, J Virol 78, 12225-35). In addition, the Ala30 to Pro mutation reduces the inhibitory activity of NS2A in induction of IFN-β promoter-driven transcription compared to that observed for the wt NS2A protein. The resulting KUN replicon with the NS2A (Ala30 to Pro) mutation was designated Sp6KUNrep4PP.

The murine GMCSF sequence was amplified by PCR with High-fidelity Pfu DNA polymerase (Promega) from plasmid pEF-BOS/GMCSF (obtained from Glenn Dranoff, Dana-Farber Cancer Institute, Boston) using forward (5′-GCGGACGCGTATGCCCACGAGAGAAAGGCTAAG-3′; SEQ ID NO:2) and reverse (5′-GCGACGCGTCATTTTTGGACTGGTTTTTTGC-3′; SEQ ID NO:3) primers with incorporated MluI restriction sites (bold). The GM-CSF PCR product (without start codon, but with authentic stop codon) was cloned into the MluI restriction site of Kunjin replicon Sp6KUNrep4PP, thereby generating Sp6KUNrep4PP-GMCSF.

Virus-Like Particles (VLPs) containing Sp6KUNrep4PP-GMCSF replicon RNA were produced in a tetracyclin-inducible packaging BHK cell line (tetKUNCprME) essentially as described previously (Harvey et al., 2004, J Virol 78, 531-538 and International Application PCT/AU2004/000752).

Briefly, Sp6KUNrep4PP-GMCSF replicon RNA was transcribed in vitro from linearized plasmid DNA with SP6 RNA polymerase and was transfected into the tetKUNCprME packaging cells by electroporation. Doxycycline was removed from the medium to allow expression of KUN structural proteins C, prM and E, which subsequently package the replicon RNA into VLPs. Culture fluids were harvested repeatedly for up to 10 days and were assayed on VERO cells to determine Sp6KUNrep4PP-GMCSF VLP titres.

### KUN GMCSF VLP Tumour Immunotherapy 1

### Introduction

To evaluate the potential for KUN VLP GMCSF gene therapy, B 16 tumours were established on syngeneic C57BL/6 mice and were treated by intra/peri-tumoural (i.t./p.t.) injections. The controls included the medium in which the VLPs are prepared and stored, and an empty VLP which did not contain the PP mutations or code for any heterologous gene.

### Methods

C57BL/6 mice where given 10⁶ B16 melanoma cells s.c. onto the shaved back. The B16 cells were in logarithmic growth in T25 flasks and were trypsined, washed once and injected in 100 ul of RPMI1640 supplemented with 10% FCS. After 4 days animals were randomly assigned into 4 groups;

1. A control group that was injected i.t./p.t with 40-50 ul of medium comprising RPMI1640 supplemented with 5% FCS;
2. A control KUN VLP group that was injected i.t./p.t. with 40-50 ul* of KUN VLP empty (Sp6KUNRep6LAEmpty) 1.7×10⁶ IU/tumour;
3. The KUN VLP GMCSF group that was injected i.t./p.t with 40-50 ul* of KUN VLP GMCSF (Sp6KUNrep4PPGMCSF) 1.7×10⁶ IU/tumour;
4. A control group that received no treatment. *The volume was adjusted so that the dose remained identical as several batches of VLPs have slightly differing titres.

The tumours were monitored as described (Anraku et al., 2002, J. Virol. 76 3791-9). Treatment occurred d0, d1, d2, d6, d7 and d 8.

### Results

The average tumour size 4 days after inoculation (which also represents the first day of treatment or 0 days after treatment initiation) for Group 1 was 12.2±SD 5.1, for Group 2 10.5±1.6, Group 3 13.2±6, Group 4 12.6±3.8. Animals were killed when the tumour reached 100 mm2 and animal survival shown as Kaplan Meier curves (FIG. 1). The KUN VLP GM-CSF treated group showed significantly increased survival compared to the Sp6KUNRep6LAEmpty treated group (Log Rank statistic p=0.0061) and the untreated control group (Log Rank statistic p=0.0037). None of the control groups were significantly different from each other.

### Conclusion

KUN VLP GM-CSF i.t./p.t. treatment provides significant therapeutic anti-cancer activity in this B16 model.

### KUN GMCSF VLP Tumour Immunotherapy 2

### Introduction

To further evaluate the potential for KUN VLP GMCSF gene therapy, B16 tumours were established on syngeneic C57BL/6 mice and were treated by intra/peri-tumoural (i.t./p.t.) injections of KUN VLP GMCSF for 10 days from d 0 to d 9. The controls included a KUN VLP encoding β-galactosidase in a vector containing the PP mutations and an untreated group.

### Methods

C57BL/6 mice where given 10⁶ B16 melanoma cells s.c. onto the shaved back. The B 16 cells were in logarithmic growth in T25 flasks and were trypsined, washed once and injected in 100 ul of RPMI1640 supplemented with 10% FCS. After 2 days animals were assigned into the following groups

1. A KUN VLP GMCSF group that was injected i.t./p.t. with 40-50 ul of KUN VLP GMCSF (Sp6KUNrep4PPGMCSF) 1.7×10⁶ IU/tumour/day from d 0 to d 9, a total of 10 daily injections (n=7).
2. A control KUN VLP group that was injected i.t./p.t. with 40-50 ul of KUN VLP βgal (Sp6KUNRep3PPβgal) 1.7×10⁶ IU/tumour/day from d0 to d 9 (n=6).
3. A group receiving no treatment (n=8).

The tumours were monitored as described (Anraku et al., 2002, supra)

### Results

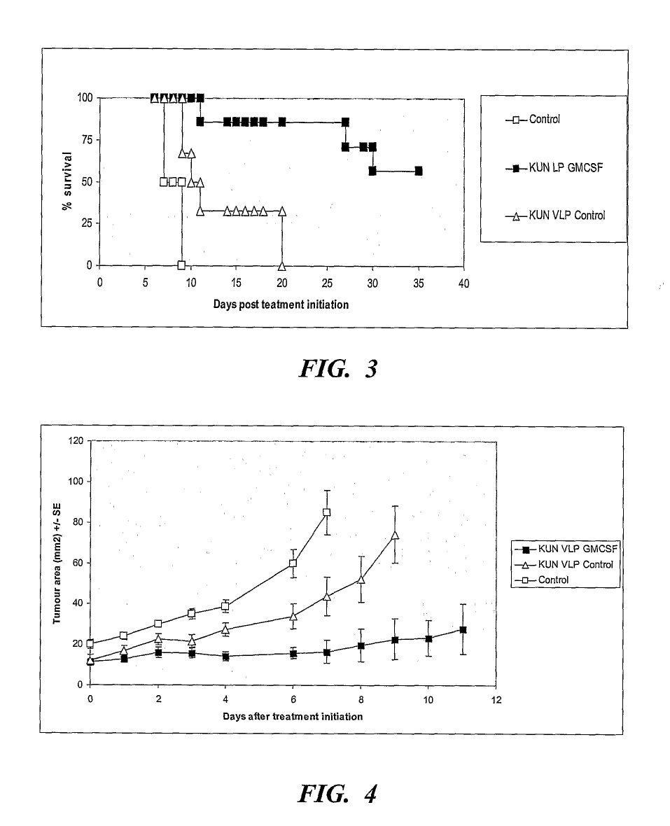

Kaplan-Meier plot of survival illustrate that 10 daily treatments with KUN VLP GMCSF significantly reduced the time to death compared with untreated animals (log rank statistic p=0.0002) or animals receiving KUN VLP Control (log rank statistic p=0.0021) (FIG. 3). The Control VLP treatment also provides some protection (log rank statistic p=0.005) compared to untreated controls (FIG. 3).

Growth curves taken until the first animal in each group was killed also showed a significant reduction in tumour growth for KUN VLP GMCSF treated animals (FIG. 4).

Perhaps most surprisingly 4 out of 7 KUN VLP GMCSF treated animals the B16 tumour became undetectable at d 27-29 and remained undetectable till the end of the current monitoring period (d 35).

### Conclusion

KUN VLP GMCSF treatment provides significant therapeutic anti-cancer activity in this B 16 model. Ten daily injections caused not only the tumour growth to be retarded, but tumours also regressed in 57% of animals, with tumours becoming undetectable 18-20 days after treatment cessation. Co administration of CpG oligonucleotides (Sharma et al., 2003, Biotechnol Lett. 25 149-53; Sfondrini et al. 2004, Cancer Immunol Immunother. 53 697-704) failed to improve this cure rate (data not shown).

The Control VLP clearly also provides some protection, presumably via IFNα/β induction.

### KUN GMCSF VLP Tumour Immunotherapy3

### Introduction

To further evaluate the potential for KUN VLP GMCSF gene therapy, B16-OVA tumours (B16 cells stably expressing ovalbumin; Anraku et al., 2002, supra) were established on syngeneic C57BL/6 mice and were treated by intra/peri-tumoural (i.t./p.t.) injections of KUN VLP GMCSF for 10 days from d 0 to d 9. The controls included a KUN VLP encoding P-galactosidase in a vector containing the PP mutations, and an untreated group. To determine whether the KUN VLP GMCSF gene therapy could synergise with therapeutic vaccination a further group was included that was vaccinated with KUN VLP encoding the murine polytope (KUN VLP mpt), which includes the ovalbumin epitope, SIINFEKL (Anraku et al., 2002, supra). KUN VLP mpt can slow B16-OVA growth when used prophylactically (Anraku et al., 2002, supra), and can slow B16-OVA growth and delay death when used therapeutically (data not shown).

### Methods

C57BL/6 mice where given 10⁶ B16 melanoma cells s.c. onto the shaved back. The B16 cells were in logarithmic growth in T25 flasks and were trypsined, washed once and injected in 100 ul of RPM11640 supplemented with 10% FCS. After 3 days animals were assigned into the following groups

1. A KUN VLP GMCSF group that was injected i.t./p.t. with 40-50 ul of KUN VLP GMCSF (Sp6KUNrep4PPGMCSF) 1.7×10⁶ IU/tumour/day from d 0 to d 9, a total of 10 daily injections (n=6).
2. As in I but also receiving 10⁷ pfu KUN VLP mpt i.p. on days 0, 5, and 9.
3. A control KUN VLP group that was injected i.t./p.t. with 40-50 ul of KUN VLP βgal (Sp6KUNRep3PPβgal) 1.7×10⁶ IU/tumour/day from d 0 to d 9 (n=6).
4. A group receiving no treatment (n=8).

The tumours were monitored as described (Anraku et al., 2002, supra).

### Results

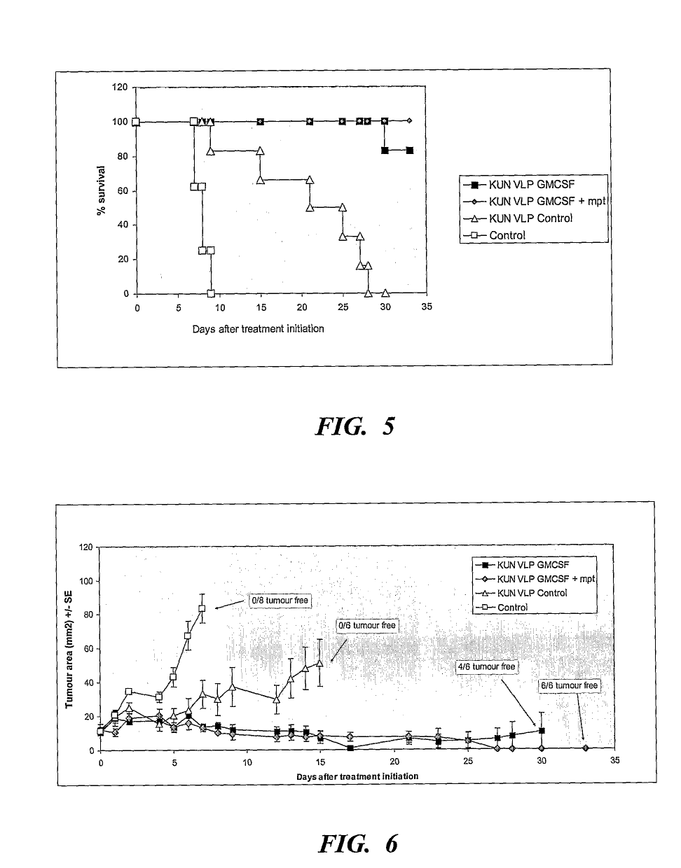

Kaplan-Meier plot of survival illustrate that 10 daily treatments with KUN VLP GMCSF significantly reduced the time to death compared with untreated animals (log rank statistic p=0.0004) or animals receiving KUN VLP Control (log rank statistic p=0.0005) (FIG. 5). The Control VLP treatment also provides protection (log rank statistic p=0.001) compared to untreated controls (FIG. 5).

The addition of KUN VLP mpt treatment to KUN VLP GMCSF therapy resulted in 6/6 mice regressing their tumours and becoming tumour free at the end of the current monitoring period (d 33). In contrast, the group receiving KUN VLP GMCSF only 4/6 animals where tumour free at this point, with one of these animals culled on d 30.

Growth curves taken until the first animal in each group was killed also showed a significant reduction in tumour growth for KUN VLP GMCSF treated animals (FIG. 6).

### Conclusion

KUN VLP GMCSF treatment provides significant therapeutic anti-cancer activity in this B16-OVA model. Ten daily injections caused not only the tumour growth to be retarded, but tumours also regressed in 67% of animals. The data also strongly suggests that combining KUN VLP GMCSF treatment with a KUN-based cancer vaccine (KUN VLP mpt) provides synergistic anti-cancer activity, with 6/6 animal tumour free on d 33. This synergy may arise from (i) enhanced anti-cancer CD8 T cell activity arising from SIINFEKL-specific CD8 T cells, (ii) enhanced tumour inflammation due to KUN replicon specific T cells raised by KUN VLP mpt vaccination and targeting KUN VLP GMCSF infected cells and/or (iii) licensing of tumour draining dendritic cells by KUN-specific T cells. We have shown that KUN VLP vaccination can induce T cell responses specific for the replicon (data not shown).

The Control VLP clearly also provides some protection, presumably via IFNα/β induction.

### KUN GMCSF VLP Immunotherapy of Mesothelioma

### Introduction

To determine whether the KUN GMCSF VLP therapy would work for other tumour cell lines a mesothelioma, AE17 (Jackaman et al., 2003, J. Immunol. 171 5051-63) was tested.

### Methods

C57BL/6J mice were injected with 1.4×10⁶ AE17 cells/mouse sc on the back. Two days later tumour bearing mice were divided to 2 groups of n=6.

Group 1. The KUN VLP GMCSF group was injected i.t./p.t with 50 ul of KUN VLP GMCSF (Sp6KUNrep5PPGMCSF) 1.5×10⁶ IU/tumour daily from d0 to d7, then the same amount of KUN VLP GMCSF (Sp6KUNrep4PPGMCSF) on d8 and 9.

Group 2. No treatment.

### Results

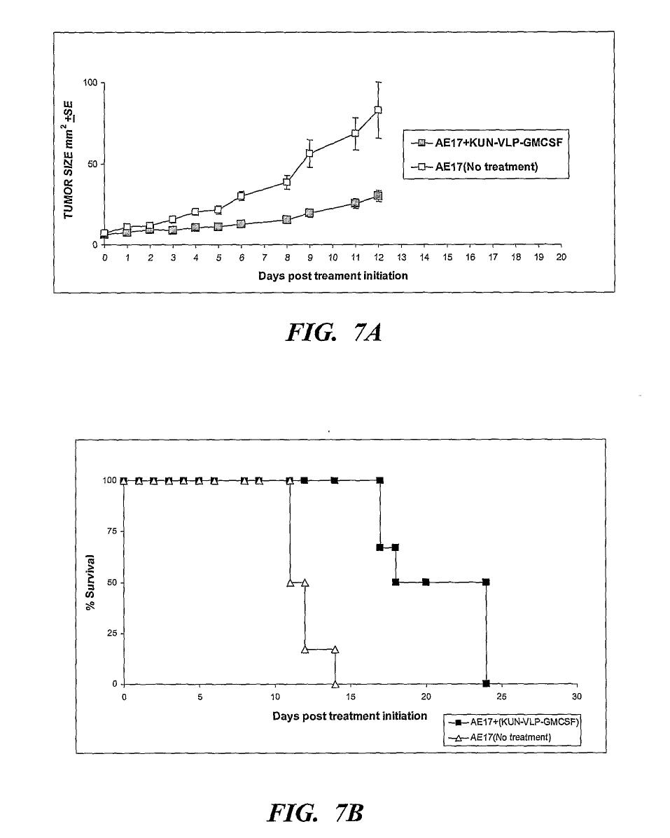

The i.t./p.t treatment with KUN VLP GMCSF of established AE17 tumours significantly (p<0.01) reduced the growth of these tumours (FIG. 7A). A Kaplan Meier curve of the same experiment is shown in FIG. 7B.

### Conclusion

This experiment indicates that KUN VLP GMCSF therapy would be effective for treatment of mesothelioma.

### KUN GMCSF VLP Immunotherapy of Colon Cancer

### Introduction

To determine whether the KUN GMCSF VLP therapy would work for other tumours a colon cancer line MC38 (Hikino et al., 2004, Anticancer Res. 24 1609-15; Tirapu et al., 2004, Int J. Cancer. 110 51-60) was tested.

### Methods

C57BL/6J mice were injected sc with 4×10⁵ MC38 cells/mouse on the shaved back. Two days later tumour bearing mice were divided to 2 groups.

Group 1 (n=6). The K(UN VLP GMCSF group was injected i.t./p.t with 50 ul of KUN VLP GMCSF (Sp6KUNrep5PPGMCSF) 1.5×10⁶ IU/tumour daily from d0 to d7, then the same amount of KUN VLP GMCSF (Sp6KUNrep4PPGMCSF) on d8 and 9.

Group 2 (n=5). No treatment.

### Results

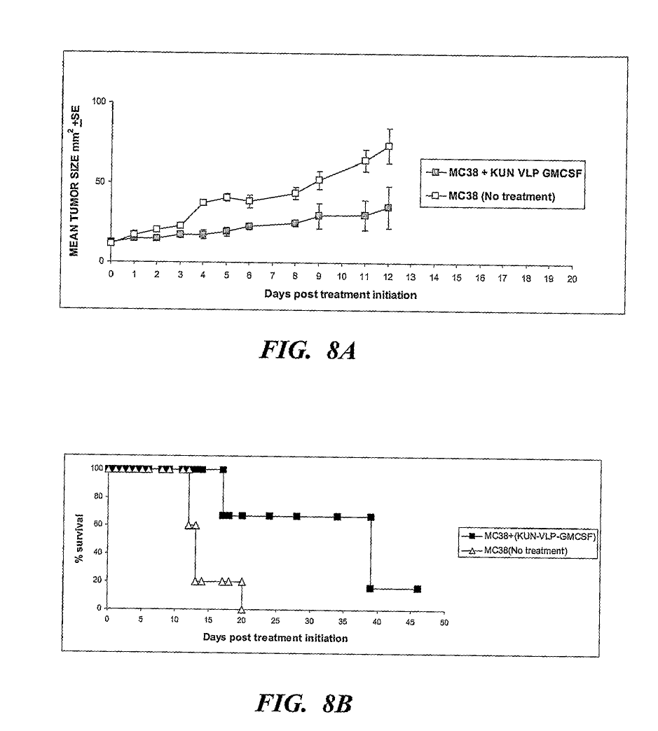

The i.t./p.t treatment of established MC38 tumours with KUN VLP GMCSF significantly (p<0.01) reduced their growth (FIG. 8A). A Kaplan Meier curve of the same experiment is shown in FIG. 8B.

### Conclusion

This experiment indicates that KUN VLP GMCSF therapy would be effective for treatment of colon cancer.

### KUN GMCSF VLP Immunotherapy of Mammary Adenocarcinoma

### Introduction

To determine whether the KUN GMCSF VLP therapy would work for other tumours, a mammary adenocarcinoma, TUBO was tested (Varadhachary et al., 2004, Int J. Cancer. 111 398-403).

### Methods

Balb/c mice were injected with 1×10⁵ TUBO cells/mouse sc on the shaved back. Seven days later tumour bearing mice were divided to 2 groups.

Group 1. (n=4) The KUN VLP GMCSF group was injected i.t./p.t with 50 ul of KUN VLP GMCSF (Sp6KUNrep5PPGMCSF) 1.5×10⁶ IU/tumour daily from d0 to d3, then the same amount of KUN VLP GMCSF (Sp6KUNrep4PPGMCSF) from day 4-8.

Group 2. (n—6) No treatment.

### Results

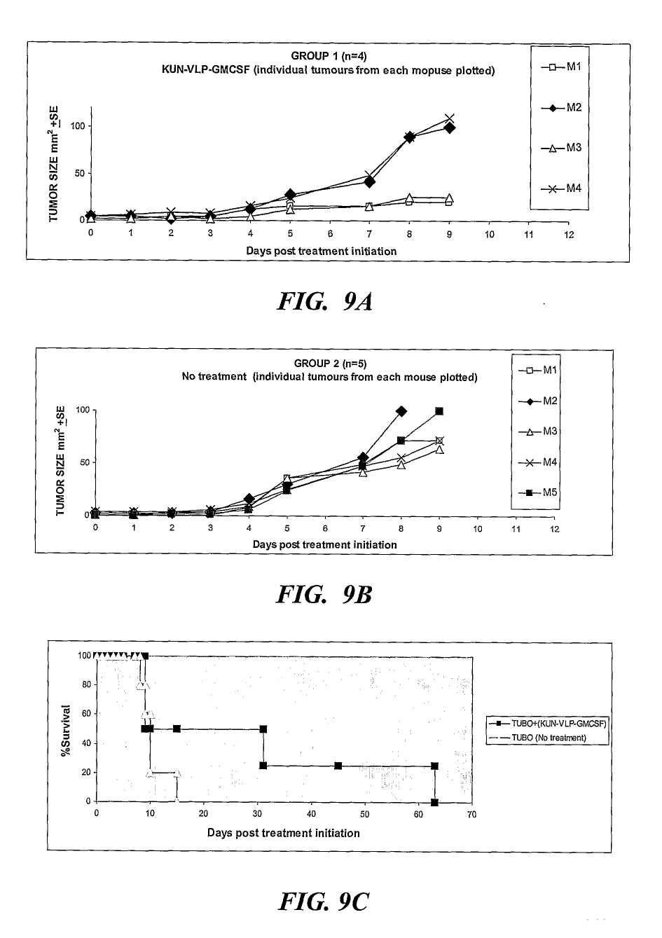

The i.t./p.t treatment of established TUBO tumours with KUN VLP GMCSF significantly (p<0.01) slowed the growth of 50% of the tumours (FIG. 9A, Group 1 white square and yellow triangle). A Kaplan Meier curve of the same experiment is shown in FIG. 9C.

### Conclusion

This experiment indicates that KUN VLP GMCSF therapy would be effective for treatment of breast cancer.

### KUN GMCSF VLP Immunotherapy of Breast Cancer

### Introduction

To determine whether the KUN GMCSF VLP therapy would work for other tumours a breast cancer line, 4T1 was tested.

### Methods

Balb/c mice were injected with 4×10⁵ 4T1 cells/mouse sc on the shaved back. Two days later tumour bearing mice were divided to 2 groups.

Group 1. (n=6) The KUN VLP GMCSF group was injected i.t./p.t with 50 ul of KUN VLP GMCSF (Sp6KUNrep4PPGMCSF) 1.5×10⁶ IU/tumour daily from d0 to d4, then the same amount of KUN VLP GMCSF on d7 and 8.

Group 2. (n=5) No treatment.

### Results

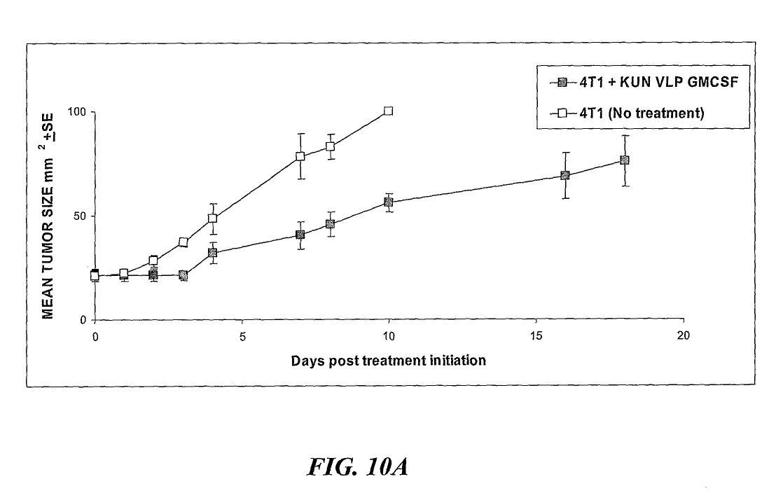

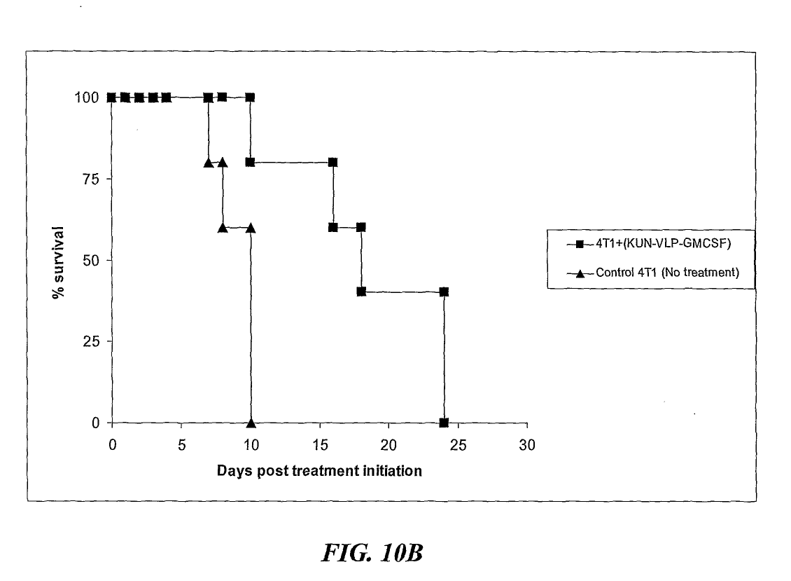

The i.t./p.t treatment of established 4T1 tumours with KUN VLP GMCSF significantly (p<0.01) reduced their growth (FIG. 10A). A Kaplan Meier curve of the same experiment is shown in FIG. 10B.

### Conclusion

This experiment indicates that KUN VLP GMCSF therapy would be effective for treatment of breast cancer.

### Production of GMCSF by KUN Replicon RNA

To confirm the production of GMCSF by the KUN RNA, which was subsequently used to manufacture KUN GMCSF VLPs, RNA was transfected into BHK by electroporation (25 uF, 1500 V, 2 pulses 10 sec apart) as described previously (Khromykh et al., 1998, J. Virol. 72 5967-77), or into B16 cells by electroporation (960 uF, 250 V, 1 pulse). The cells were seeded at 1.25×10⁵ cells per well of a 24 well plate and were incubated in standard medium for 3 days. Approximately 10-30% of cells were transfected as determined by IFA. The duplicate or triplicate supernatants were then assayed using a murine GMCSF ELISA assay kit (BD Biosciences) and biological activity was assayed using serially diluted samples and the GMCSF/IL-3 responsive FDCP1-1 cell line (Naparstek et al., 1986, Blood 67 1395-1403).

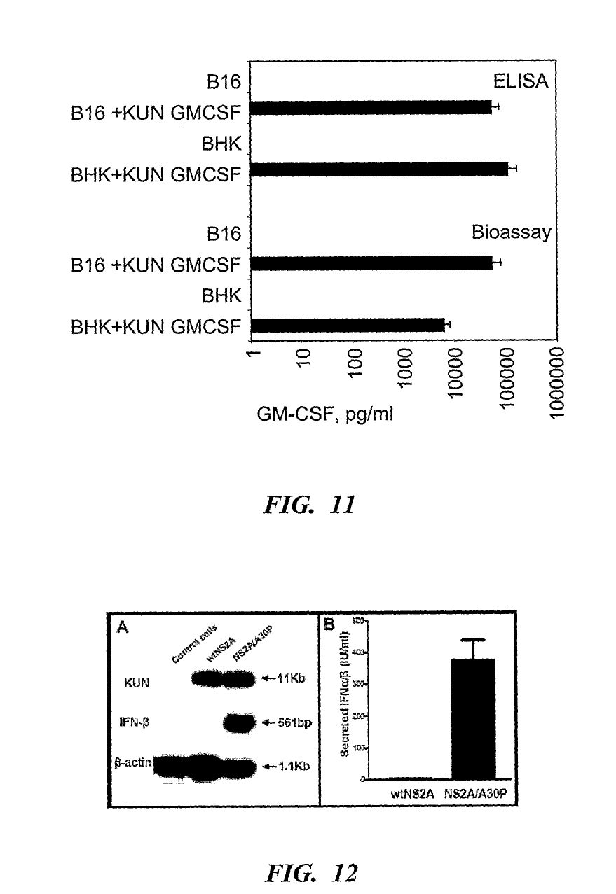

As shown in FIG. 11, both assays illustrated that BHK and B 16 cells transfected with KUN GMCSF RNA produced 10-100 ng/ml of GMCSF over 3 days. It should be noted that cell division occurs during this period and when a KUN transfected cell divides both daughter cells will contain KUN RNA and will produce GMCSF (Varnavski et al., 1999, Virology 255 366-75).

### IFN-β mRNA Transcription and Production of Secreted IFN-α/β by the Wild Type KUN Virus and KUN Virus with Ala30 to Pro Mutation in NS2A

In order to compare the efficiency of the wt and NS2A-mutated KUN viruses in induction of IFN-β transcription, total RNA from A549 cells infected for 24 h with MOI of 1 of the wild type KUN virus and MOI of 3 of the NS2A-mutated KUN virus each virus was subjected to the Northern blot hybridization with the probes specific for IFN-β mRNA, KUN RNA and β-actin mRNA. The results showed that, the amount of IFN-β mRNA in cells infected with NS2A-mutated KUN virus was ˜6-fold higher than that observed in cells infected with the wt KUN virus (see FIG. 11A). Note that the amount of KUN RNA was similar for the wild type and the mutant virus at the time of testing (24h, FIG. 12A). Testing the 24h culture fluid from infected cells for the presence of IFN-α/β by bioassay (Antalis et al., 1998, J Exp Med., 187 1799-811) showed that NS2A-mutated KUN induced production of much higher amounts of IFN-α/β than the wt KUN (FIG. 12B).

The sensitivity of the assay (˜7.81 U/ml of reference IFN-α provided 50% protection of A549 cells from SFV challenge) did not allow for the detection of any biologically active IFN-α/β in culture fluid of A549 cells infected with the wild type virus, while ˜370 IU/ml of biologically active IFN-α/β was detected in cells infected with the NS2A-mutated virus. These results demonstrate two major novel findings: (i) the induction and secretion of IFN-α/β is inhibited by the wild type KUN virus, and (ii) a single Ala to Pro amino acid substitution at the position 30 of the NS2A protein increased induction and secretion of IFN-α/β.

### Infection of Tumour Cells by KUN Replicon VLPs

VLPs encoding β-gal were manufactured and aliquoted in small aliquots and stored in RPMI 1640 supplemented with 10% FCS and 10 mM HEPES at −70° C. A panel of tumour cells were grown on cover slips over night and were infected with 300 ul of KUN VLP suspended in RPMI with 2% FCS and 10 mM HEPES at a MOI of 10 using Sp6KUNrep3PAβgal or Sp6KUNrep2LAEmpty. The 24 well plates were placed into the incubator and rocked every hour. After the 3 h incubation the wells were toped up with 1 ml of medium and the cell cultured for a further 60 h. After 60 h the cells were washed briefly and fixed in cold acetone/methanol (50/50) for 2 mins. The cover slips were then washed, blocked and stained with a rabbit polyclonal anti-KUN NS3 antisera (used at 1/500) and an FITC labeled secondary antibody. The cells were examined under a fluorescence microscope and the number of uninfected (phase visible) and infected (fluorescent) cells in 10 representative fields using a 20× objective were counted and a percentage calculated.

Table 1 illustrates that KUN replicon VLPs are able to infect a large number of different cancers thus we envisage that KUN replicon VLPs encoding cytokines like GMCSF would be able to find utility in treating a wide variety of different cancers.

We also have some evidence that the percentage infection may be higher in cells grown on plastic compared with cells grown on glass (as in Table 1). For instance B16 cells grown on plastic and infected with MOI 10 as above show >40-70% infection.

### Infection of Tumour Cells with Kunjin VLPs Containing Replicon RNA of New York 99 Strain of West Nile Virus

The West Nile replicon construct with deletion of greater than 92% of the structural region was generated by P.-Y. Shi (USA) from the full-length clone of New York isolate of West Nile virus described previously Shi et al., 2002. Virology 296 219-33.). Electroporation of WN replicon RNA into packaging cell line tetKUNCprME (Harvey et al., 2004, supra) followed by the induction of expression of KUN structural genes C, prM, and E by removal of doxycycline resulted in production of 7×10⁷ IU/ml of secreted VLPs by 4d post-electroporation. Thus, the VLPs contain WN replicon RNA packaged by the Kunjin structural proteins C, prM, and E. Electroporation of KUN replicon RNA RNAleu performed in the parallel experiment resulted in production of comparable titres (108 IU/ml) of VLPs (Harvey et al., 2004, supra).

VLPs containing Kunjin or WN replicon RNAs were used to infect Lewis Lung and TC-1 tumour cells at multiplicity of infection equal to 10. The efficiency of infection was analysed by immunofluorescence analysis with cross-reacting antibodies to Kunjin NS3 protein. Table 2 shows that the efficiency of infection with VLPs containing WN replicon RNA was greater that that obtained in cells infected with VLPs containing Kunjin replicon RNA.

Thus, we envisage that construction of West Nile replicons encoding GMCSF may allow improved efficiency of infection of some tumour cells in vitro. There may be a correlation between the ability of KUN VLPs to infect the tumour cells in vitro and the ability of KUN GMCSF VLP therapy to provide effective cancer therapy in vivo. We further envisage that replicons constructs can be selected for replication in tumour cells and thereby provide mutations, which might improve the ability of the replicon system to produce GMCSF in tumour cells in vivo.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, patent and scientific literature referred to in this specification are incorporated herein by reference in their entirety.

TABLE 1

| Cancer cell line | % cells infected |
|---|---|
| Vero/BHK | 80-100% |
| B16 melanoma | 10.5-25% |
| Lewis Lung carcinoma | 2.9-9.1% |
| HeLa cervical carcinoma | 32% |
| A549 lung epithelial carcinoma | 16.1% |
| DU145 prostate cancer | 2.8% |
| MCF7 Breast cancer | 2.9% |
| ACHN human renal carcinoma | 66% |
| Colo205 colon cancer | 1.1% |
| TC-1 epithelial (E6, E7, c-Ha-ras) | 4.2-6.9% |
| AE17 mesothelioma | 11-17% |

TABLE 2

| | | % infected |
|---|---|---|
| Tumour line | % infected by KUN replicon VLPs | by WN replicon VLPs |
| Lewis Lung | 3.1, 9.1, 2.9 (three expts). | 48 |
| TC-1 | 4.2, 6.9 | >30. |

1. A flavivirus replicon construct comprising a nucleotide sequence encoding:
(i) a flavivirus replicon that is incapable of producing infectious virus; and
(ii) granulocyte macrophage colony stimulating factor (GMCSF);
wherein the nucleotide sequence in (i) encodes a flavivirus replicon having one or more amino acid mutations, deletions or substitutions in a non-structural protein of said replicon, which in an animal cell, enhance induction of IFNα/β compared to a wild-type flavivirus replicon-encoded non-structural protein.

2. The flavivirus replicon construct of claim 1, wherein said non-structural protein is selected from the group consisting of: NS2A, NS2B, NS3, NS4A and NS4B.

3. The flavivirus replicon construct of claim 2, wherein said one or more amino acid mutations, deletions or substitutions in said flaviviral non-structural protein is/are selected from the group consisting of:
(I) a mutation of Alanine 30 to Proline in NS2A; and
(II) a mutation of Asparagine 101 to Aspartate or Glutamate in NS2A.

4. The flavivirus replicon construct of claim 1, which encodes a Kunjin virus replicon.

5. An expression construct comprising the flavivirus replicon construct of claim 1 operably linked to one or more regulatory sequences.

6. The expression construct of claim 5, wherein said non-structural protein is selected from the group consisting of: NS2A, NS2B, NS3, NS4A and NS4B.

7. The expression construct of claim 6, wherein said one or more amino acid mutations, deletions or substitutions in said flaviviral non-structural protein is/are selected from the group consisting of:
(I) a mutation of Alanine 30 to Proline in NS2A; and
(II) a mutation of Asparagine 101 to Aspartate or Glutamate in NS2A.

8. The expression construct of claim 4, which encodes a Kunjin virus replicon.

9. The expression construct of claim 4 which is in DNA form, wherein the one or more regulatory sequences include a promoter.

10. The expression construct of claim 9, which facilitates transcription of flavivirus replicon-encoding RNA in vitro.

11. The expression construct of claim 10, wherein the promoter is a T7 or SP6 promoter.

12. The expression construct of claim 9, which facilitates transcription of flavivirus replicon-encoding RNA in an animal cell.

13. The expression construct of claim 12, wherein the promoter is a CMV promoter.

14. The expression construct of claim 12, wherein the promoter is a regulatable promoter.

15. The expression system of claim 14, wherein the regulatable promoter is a tetracycline-regulatable promoter.

16. An expression system comprising:
(i) an expression construct according to claim 4; and
(ii) a packaging construct that is capable of expressing one or more proteins that facilitate packaging of said expression vector or construct into flavivirus virus like particles (VLPs) by said packaging cell.

17. The expression system of claim 16, wherein the expression construct is in RNA form.

18. The expression system of claim 17, wherein the RNA has been transcribed in vitro.

19. The expression system of claim 16, wherein the expression construct is in DNA form.

20. The expression system of claim 19, wherein the expression construct further comprises a promoter operable in said packaging cell to facilitate expression of a flavivirus replicon-encoding RNA by the packaging cell.

21. The expression system of claim 20, wherein the promoter is a regulatable promoter.

22. The expression system of claim 21, wherein the regulatable promoter is a tetracycline-regulatable promoter.

23. The expression system of claim 22, wherein the regulatable promoter is operably linked to a nucleotide sequence encoding a flavivirus structural protein translation product, which comprises C protein, prM protein and E protein.

24. A flavivirus virus like particle (VLP) comprising the replicon construct of claim 1 in RNA form.

25. A packaging cell comprising the expression system of claim 16.

26. The packaging cell of claim 25, which is a BHK21 cell.

27. A pharmaceutical composition comprising a VLP that comprises the replicon construct of claim 1, together with a pharmaceutically-acceptable carrier, diluent or excipient.

28. A pharmaceutical composition comprising the expression construct of claim 12 together with a pharmaceutically-acceptable carrier, diluent or excipient.

29. A method of prophylactic or therapeutic treatment of a tumour or cancer in an animal, said method including the step of administering flavivirus replicon construct of Claim 1 to an animal to thereby reduce, arrest, eliminate or otherwise treat the tumour or cancer in said animal.

30. The method of claim 29, wherein the flavivirus replicon construct is in RNA form.

31. The method of claim 30, wherein the flavivirus replicon construct is in a VLP.

32. The method of claim 29 wherein the flavivirus replicon construct encodes a Kunjin virus replicon.

33. A method of prophylactic or therapeutic treatment of a tumour or cancer in an animal, said method including the step of administering flavivirus expression construct of claim 12 to an animal to thereby reduce, arrest, eliminate or otherwise treat the tumour or cancer in said animal.

34. The method of claim 33 when used in combination with at least one other immune-based therapy.

35. The method of claim 33, wherein the flavivirus expression construct encodes a Kunjin virus replicon.

36. The method of claim 29 which includes the step of administering the flavivirus replicon construct or the flavivirus expression construct intra-tumourally or peri-tumourally.

37. The method of claim 29, wherein the animal is a mammal.

38. The method of claim 37, wherein the mammal is a human.

39. The method of claim 29, wherein the tumour or cancer is melanoma, lung carcinoma, cervical carcinoma, lung epithelial carcinoma, prostate cancer, breast cancer, renal carcinoma, colon cancer, epithelial cancers and mesothelioma.

40. An isolated cell obtained from an animal treated according to claim 29.

41. The isolated cell of claim 40, which is an antigen-presenting cell or a lymphocyte.

42. A method of adoptive immunotherapy of a tumour or cancer in an animal including the step of administering the isolated cell of claim 41 to said animal to thereby reduce, arrest, eliminate or otherwise treat the tumour or cancer in said animal.

43. The method of claim 42, wherein the animal is a mammal.

44. The method of claim 43, wherein the mammal is a human.

45. The method of claim 42, wherein the tumour or cancer is melanoma, lung carcinoma, cervical carcinoma, lung epithelial carcinoma, prostate cancer, breast cancer, renal carcinoma, colon cancer, epithelial cancers and mesothelioma.

46. The method of claim 33, which includes the step of administering the flavivirus replicon construct or the flavivirus expression construct intra-tumourally or peri-tumourally.

47. The method of claim 33, wherein the animal is a mammal.

48. The method of claim 33, wherein the tumour or cancer is melanoma, lung carcinoma, cervical carcinoma, lung epithelial carcinoma, prostate cancer, breast cancer, renal carcinoma, colon cancer, epithelial cancers and mesothelioma

49. An isolated cell obtained from an animal treated according to claim 33.
